(19) United States
(12) Patent Application Publication
(10) Pub. No.: US 2023/0398016 A1
(43) Pub. Date: Dec. 14, 2023

(54) MATERIALS AND METHODS OF MANUFACTURING RELATED TO APPARATUS FOR COLLECTING URINE

(71) Applicant: ACS Industries, Inc., Lincoln, RI (US)

(72) Inventors: George Greenwood, Tiverton, RI (US); Mark Pighills, McAllen, TX (US)

(73) Assignee: ACS Industries, Inc., Lincoln, RI (US)

(21) Appl. No.: 18/449,664

(22) Filed: Aug. 14, 2023

Related U.S. Application Data
(63) Continuation-in-part of application No. 18/187,629, filed on Mar. 21, 2023.
(60) Provisional application No. 63/322,249, filed on Mar. 22, 2022.
(60) Provisional application No. 63/371,392, filed on Aug. 14, 2022.

(51) Int. Cl. A61F 5/44

(52) U.S. Cl. CPC A61F 5/4405

(57) ABSTRACT

A device for collecting urine comprises a permeable core assembly, an exterior housing and a suction tube assembly. The core assembly has a tubular fluid permeable core with a fluid permeable wicking cover. The exterior housing is capsular-shaped and has a reservoir at a lower end, an outlet port at an upper end, and an elongated window opening. The core assembly is received within the housing with the core assembly extending across the elongated window. The suction tube assembly extends through an interior of the core assembly and has a first end disposed in the reservoir and a second end exiting the outlet port. The suction tube assembly comprises an elongated shape retention element and a suction tube body having a suction lumen providing a suction path from a first end thereof to a second end thereof, and a second shape retention lumen receiving the elongated shape retention element.

### CROSS-REFERENCE TO RELATED APPLICATIONS

This application is a continuation-in-part of U.S. application Ser. No. 18/187,629 filed Mar. 21, 2023, which claims the benefit of U.S. Provisional Application No. 63/322,249, filed Mar. 22, 2022, the entire contents of which is incorporated herein by reference

This application also claims the benefit of U.S. Provisional Application No. 63/371,392, filed Aug. 14, 2022, the entire contents of which is incorporated herein by reference.

### BACKGROUND OF THE DISCLOSURE

The present disclosure relates generally to external urinary collection apparatus. Disabled or injured persons, or persons with restricted movement during long periods of time, i.e. pilots, may have an impaired or limited mobility that prevents them from urinating in a sanitary manner.

External urinary collection apparatus are known in the art. U.S. patent Ser. No. 10/226,376 discloses and describes a well known external urinary collection apparatus which has wide industry acceptance. The entire contents and disclosure of USP10226376 are incorporated herein by reference.

As with all high-volume medical products there are always continuing efforts to improve the product and materials, improve manufacturing techniques, and to reduce costs.

### SUMMARY OF THE DISCLOSURE

The present disclosure provides improvements related to the following:
the addition of a Zeolite material into the internal core material as an ammonia absorbing material;
the placement of a shape retention element within a secondary lumen in the suction tube body;
providing the shape retention element with external embossed formations to prevent longitudinal slippage within the suction tube body;
providing a D-shaped suction tube body to allow for additional cushioning material adjacent to the wearer's body;
creating the core material using multiple different denier fibers and a rando-webber air lay process to concentrate finer denier fibers toward a top (outer) surface for a softer feel; and
providing the finished core with a ribbed outer surface through a blossoming process of heating and cooling the core while trapped in a confining curved mold with internal longitudinal ribs.

### BRIEF DESCRIPTION OF THE DRAWING FIGURES

Exemplary embodiments will now be described further by way of example with reference to the following examples and figures, which are intended to be illustrative only and in no way limiting upon the scope of the disclosure.

FIG. 1 is a perspective view of an exemplary device in accordance with the teachings of the present invention;

FIG. 2 is an exploded perspective view thereof;

FIG. 3 is a perspective view of the core assembly thereof;

FIG. 4 is a cross-sectional view thereof taken along line 4-4 of FIG. 3;

FIG. 5 is a perspective view of another exemplary device in accordance with the present disclosure;

FIG. 6 is an exploded perspective view thereof;

FIG. 7 is a plan view of the suction tube assembly;

FIG. 8 is a perspective view of the shape retention element (wire);

FIG. 9 is a cross-sectional view of the suction tube assembly taken along line 9-9 of FIG. 7;

FIG. 10 is another cross-sectional view thereof with an alternate D-shaped suction tube body;

FIG. 11 is a cross-sectional view of the device taken along line 11-11 of FIG. 5;

FIG. 12 is a perspective view of a blossoming mold and shaping rod in accordance with the teachings of the present invention.

### DESCRIPTION OF THE EXEMPLARY EMBODIMENTS

Certain exemplary embodiments will now be described to provide an overall understanding of the principles of the structure, function, manufacture, and use of the device and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present disclosure. Further, in the present disclosure, like-numbered components of the embodiments generally have similar features, and thus within a particular embodiment each feature of each like-numbered component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Further, to the extent that directional terms like top, bottom, up, or down are used, they are not intended to limit the systems, devices, and methods disclosed herein. A person skilled in the art will recognize that these terms are merely relative to the system and device being discussed and are not universal.

The present disclosure generally provides improvements related to the following features and methods of manufacturing or assembly of a urine collecting device:
(1) improved wicking materials for use in the external permeable membrane (outer bandage covering);
(2) the placement of a forming wire within the core wherein the forming wire may have rolled ends to prevent any sharp ends; and
(3) improved production and assembly methods related to continuous processing of the external covering and core, continuous seaming or sewing of the core assembly, automatic cutting to length during production, vacuum hold for insertion of the core into the outer impermeable covering, and heat mold glueing/forming of the outer housing to the inner core.

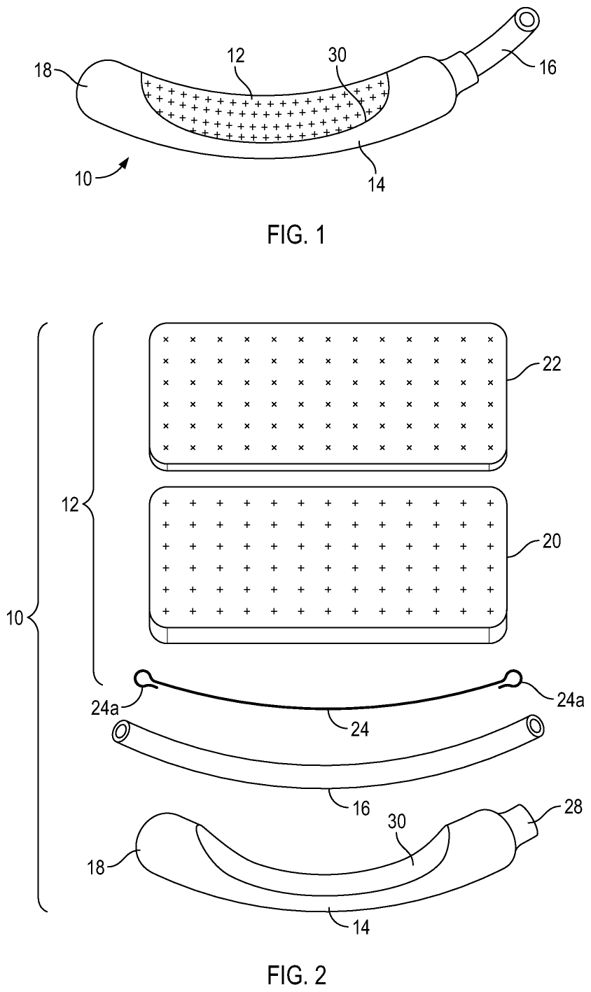

Referring to FIGS. 1 and 2 a device for collecting urine is illustrated and generally indicated at 10. The device 10 comprises a fluid permeable core assembly 12, an elongated capsular-shaped, fluid impermeable housing 14, and a suction tube 16.

In an exemplary use, the urine collection device 10 is arranged in a generally vertical position in contact with or close proximity to the subject's urethra, such that a fluid, such as urine, can be absorbed by the permeable core assembly 12, flow through the permeable core assembly and, by gravity, flow downwardly into a housing reservoir 18 where it can be removed through the suction tube 16. The general construction and function of the device 10 is known and described in USP 10226376 and therefore the broader aspects of the device will not be discussed in detail.

Referring to FIGS. 2-4, the permeable core assembly 12 comprises a tubular, fluid permeable core 20 having a generally cylindrical external surface, a fluid permeable wicking cover 22 disposed on the external surface of the tubular fluid permeable core 20, and may also include an elongated shape retention element 24 extending along a longitudinal extent of the core, the shape retention element 24 being entirely imbedded within the core 20.

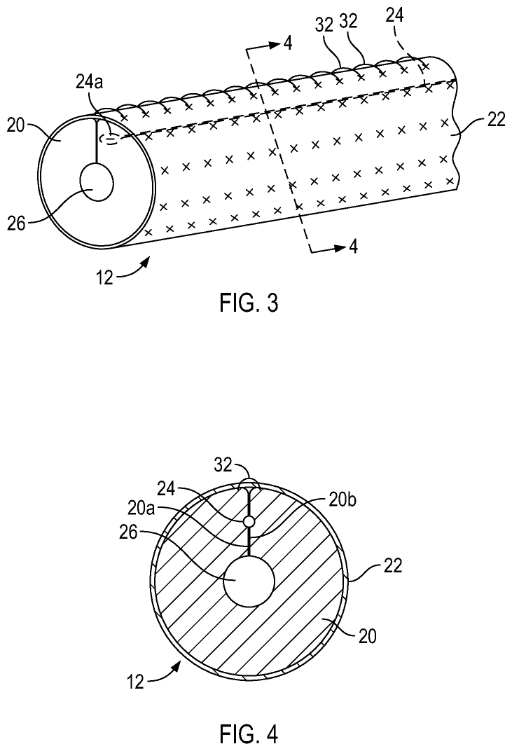

The fluid permeable core 20 may be a non-woven fibrous bio-compatible polymer material (such as non-woven nylon or polyester) which is produced as a flat slab, or bat, of material (batting) (See FIG. 2) with thickness and planar dimensions which will result in, when rolled and seamed at the side edges, a cylindrical core (FIGS. 3-4) having a diameter of approximately 1.0 to 2.5 inches. The rolled material may form an internal axial lumen 26 extending longitudinally through the center of the core.

The fluid permeable wicking cover 22 is disposed on the outer surface of the core 20 and comprises a bio-compatible natural fiber material, which may in some embodiments comprise at least one layer of non-woven bamboo material. In some embodiments, the wicking cover may comprise two-layers of bamboo material (2-ply). Non-woven, natural bamboo fiber material has been tested and shown to provide a superior wicking or capillary action to draw urine away from the user's skin into the core and thus provide improved comfort with less skin irritation.

The shape retention element 24 may comprise a wire or other formed material which may retain a predefined arcuate curvature which is suitable for the intended anatomy of the end user. In this regard, the wire 24 may be pre-shaped with a specific arcuate curvature. In the case of a pre-shaped wire, it is also preferable that the wire ends be rolled over or formed into eyelets 24A so that wire end turned back inwardly and the terminal end of the element has a rounded surface to prevent piercing through the device and injuring the user. In some embodiments, the shape retention element 24 may be formed from a ductile material which can be bent into a predefined curvature at the time of use.

To further prevent injury to the user, the shape retention element 24 may be imbedded within the core material 20 during formation of the batted core material or may be inserted and imbedded between the side edges of the core material during seaming of the edges to form the core (see FIGS. 3-4).

The capsular-shaped housing 14 may be molded or formed from a water or fluid impermeable pliable polymer or other pliable material. The housing 14 may have a reservoir 18 at a lower end for collecting a volume of liquid, or urine, that has been wicked inwardly to the core assembly 12 and that has flowed downwardly by gravity towards the lower end of the device 10. The housing 14 may further comprise an outlet port 28 at an upper end thereof through which collected urine may be removed through the suction tube 16.

The housing 14 still further includes an elongated collection window 30 extending at least partially between the lower reservoir end and the upper outlet port end. The core assembly 12 is received within the housing 14 such that the core assembly is disposed within and extends across the elongated window 30.

The suction tube 16 may generally be formed from a flexible PVC or urethane tubing material and be of a sufficient length such that it may extend through the interior lumen 26 of the core assembly 12 with a first end disposed in the reservoir and a second end exiting the housing 14 through the outlet port 28 where it can be attached to a vacuum source (not shown).

In some embodiments, the core assembly 12 may be adhered to an interior of the housing 14 during assembly. It has been found that in some cases, consumers may be reusing the housings 14 with new core materials to save costs. This is not a recommended activity due to the potential for unsanitary conditions if proper cleaning and disinfecting of the housing is not provided. Adhering the core assembly 12 to the housing 14 prevents the consumer from disassembling the device and replacing the internal components for re-use and creates a more sanitary single-use device.

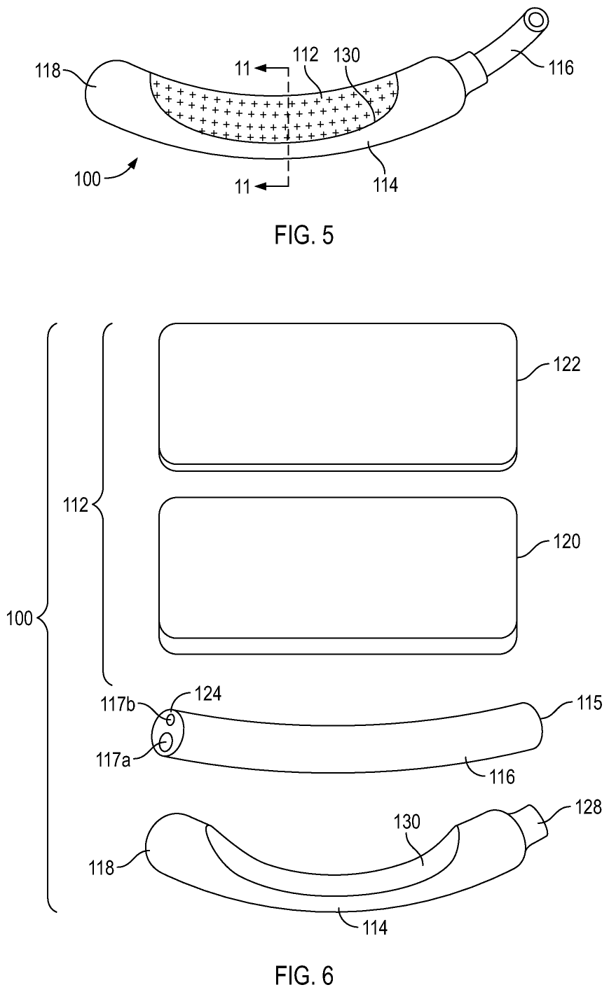

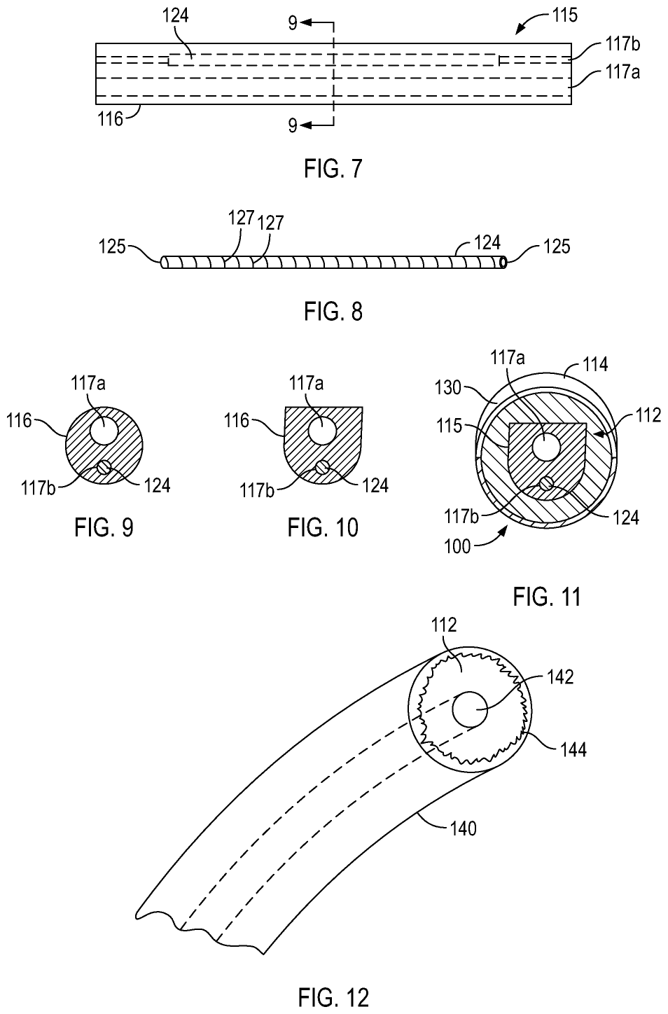

Referring to FIGS. 5-11 another exemplary device for collecting urine is illustrated and generally indicated at 100.

The device 100 comprises a fluid permeable core assembly 112, an elongated capsular-shaped, fluid impermeable housing 114, and a suction tube assembly 115.

As described hereinabove, the capsular-shaped housing 114 may be molded or formed from a water or fluid impermeable pliable polymer or other pliable material. The housing 114 may have a reservoir 118 at a lower end for collecting a volume of liquid, or urine, that has been wicked inwardly to the core assembly 112 and that has flowed downwardly by gravity towards the lower end of the device 100. The housing 114 may further comprise an outlet port 128 at an upper end thereof through which collected urine may be removed through the suction tube assembly 115. The housing 114 may still further includes an elongated collection window 130 extending at least partially between the lower reservoir end and the upper outlet port end. The core assembly 112 is received within the housing 114 such that the core assembly is disposed within and extends across the elongated window 130.

The suction tube assembly 115 comprises an elongated shape retention element 124, and a suction tube body 116 having a suction lumen 117A providing a suction path from a first end thereof to a second end thereof, and a second shape retention lumen 117B receiving the elongated shape retention element 124 therein.

The suction tube body 116 may generally be formed from a flexible PVC or urethane tubing material and be of a sufficient length such that it may extend through an interior lumen 126 of the core assembly 112 with a first end disposed in the reservoir and a second end exiting the housing 114 through the outlet port 128 where it can be attached to a vacuum source (not shown).

The shape retention element 124 may be formed with a predefined rigid curvature or may comprise a ductile metal which is bendable to a predefined shape during use. In order to prevent potential injury, the shape retention element 124 is provided with rounded or semispherical terminal ends 125. These rounded ends 125 are provided during a cutting and forming process for each shape retention element.

In an exemplary assembly method, the retention element 124 may be formed to be about 6 inches in length, where the entire suction tube body 116 may be about 10 inches in length. The purpose of a shorter retention element 124 is to prevent the end(s) of the retention element from extending beyond the end of the suction tube body 116 where it could possibly contact the wearer. In this regard, a conventional co-extrusion method would not be suitable for imbedding the retention element (wire) 124 within the suction body 116 as the retention wire would extend along the entire length of the suction tube body and potentially protrude from the end when the shape retention element is curved into a final shape. In this regard, a novel insertion method is disclosed where a 0.080″ diameter retention wire 124 (6 inches in length) may be inserted into the longitudinal center area of a smaller 0.050″ diameter lumen 117B (10 inches in length) by forcing high pressure air through the lumen 117B at one end to expand the lumen and lubricate insertion of the larger diameter retention wire 124 into the smaller diameter lumen from the opposing end. Once the wire is inserted into the central location between the ends, the air pressure is removed and the lumen shrinks back down onto the outside of the retention wire 124. The result is a near permanent fixation of the retention wire 124 in the lumen 117B where it cannot be moved out of position. In a further embodiment, the shape retention element (wire) 124 may also be provided with embossed surface features 127 (See FIG. 8) on at least a portion of the exterior surface. These surface features will imbed within the inner surface of the lumen further preventing any possible longitudinal movement of the wire within the lumen.

In some embodiments, as seen in FIGS. 10-11, the suction tube body 126 has a D-shaped cross-section with a planar front surface and a rounded back surface, wherein the suction lumen 117A is oriented adjacent the planar surface and wherein the shape retention lumen 117B is oriented adjacent the rounded surface.

When the suction tube assembly 115 is assembled within the core 112 and the outer housing 114, the planar surface of the body 116 is oriented toward the window 130 to provide a flatter engagement surface toward the user. A primary complaint of the user is a longitudinal pressure ridge that creates discomfort. By forming a flatter surface facing the body, the width of the pressure ridge or engagement area is enlarged and localized linear pressure is minimized. The flatter facing surface also allow for additional cushioning material to be located between the suction tube body 116 and the user. Such additional cushioning material may be provided by an additional layer of core material along the planar surface or in some embodiments, an additional thickness of the core material may be provided during production of the core material batting.

The permeable core assembly 112 comprises a tubular, fluid permeable core 120 having a generally cylindrical external surface, a fluid permeable wicking cover 122 disposed on the external surface of the tubular fluid permeable core 120,

The fluid permeable core 120 may be a non-woven fibrous bio-compatible polymer material (such as non-woven nylon or polyester) which is produced as a flat slab, or bat, of material (batting) (See FIG. 6) with thickness and planar dimensions which will result in, when rolled and seamed at the side edges, a cylindrical core (FIG. 11) having a diameter of approximately 1.0 to 2.5 inches. The rolled material may form an internal axial lumen 126 extending longitudinally through the center of the core 120.

The fluid permeable wicking cover 122 is disposed on the outer surface of the core 120 and comprises a bio-compatible natural fiber material, which may in some embodiments comprise at least one layer of non-woven bamboo material. In some embodiments, the wicking cover may comprise two-layers of bamboo material (2-ply). Non-woven, natural bamboo fiber material has been tested and shown to provide a superior wicking or capillary action to draw urine away from the user's skin into the core and thus provide improved comfort with less skin irritation.

In assembly, the outer soft surface of the core material 120 is overlaid with the bamboo wicking layer 122 (these materials are the same width) and then overlaid again with the Kamisoft material 132, which may be about ½ inch wider on each side, allowing the Kamisoft to be folded up the side edge wall of the ½ inch thick core. With the Kamisoft layer on the outside, the overlaid materials are folded like a taco to bring the opposing edges into contact and then heat/melt sealed along the longitudinal edge. Other polyester outer covering materials are also contemplated.

A known issue with the current commercial product is the softness or compressability of the absorbent core material 120. Because the device must be worn in close conforming engagement with the wearer, softness of the core material is critical to patient/wearer comfort. As noted above, the core material is a non-woven fiber material which is laid down in a web with a thickness of about 0.5 in and then rolled into a cylindrical tube. The outer surface of the tubular core 120 (or cover 122) that is exposed through the silicone reservoir housing 114 then presses against the wearer.

An improved core material 120 according to the invention may be formed by using multiple different denier core fiber materials and modifying a non-woven web forming process to concentrate finer (smaller) denier fibers toward a top (outer) surface of the web thus forming a softer cotton like outer surface. Denier is a unit of measurement used to determine the fiber thickness of individual threads or filaments used in the creation of textiles and other materials. Fibers with a higher denier tend to be thick, sturdy, and durable while fibers with a lower (smaller) denier tend to be sheer, soft, and silky.

Using a rando-webber (random air lay process) an 8 denier bi-component (bico) (core/sheath) fiber of Polyester (core)/HDPE (high density polyethene) (sheath) or polyester CO-PET fiber was blended with 15 denier PET (polyethylene terephthalate) fiber to form web which is approximately 0.5 inch thick. As noted above, the thicker denier fibers are concentrated toward the lower inner surface while the smaller denier fibers are concentrated toward the upper (outer) surface to form a softer cotton like surface. The thickness may vary depending on the desired outer diameter of the device. Other bico fibers and non-bico fiber materials may be substituted as appropriate for various non-woven end product outcomes.

In an exemplary production method, a rando-webber system with a perforated screen may be used to form the described core material. The system used to prepare the material includes a vacuum source to pull the fibers onto the moving screen (7). Typical airlay processes use a single source material for a homogenous web.

The airlay process for forming the present core material web differs from a typical non-woven airlay process in that two different denier fibers are utilized. Due to the natural weight and size of the different fibers, the heavier (larger) denier fibers naturally fall quicker and are collected on the screen in a higher concentration first forming the lower (inner surface layer of the web). The smaller denier bico fibers naturally tend to float in air (like feather) and are concentrated more towards the top (outer surface) creating a softer upper layer. There is mixing of the thicker and thinner denier fibers throughout the web to create an interconnected non-woven material.

The resulting web is run through a primary oven section at around 350° F. to melt bond the bico fibers throughout the web, and then bonded web is flipped and spray coated on the second side (what will become the inner side of the core) before final curing on the third level. The spray mix may comprise a carboxylated SBR, (styrene butadiene) blended with around 2% melamine formaldehyde resin to crosslink the mix. One or more spray coating passes may be used. Other crosslinking formulations may also be appropriate depending on the web materials.

This unique process leaves a soft cotton wool like feeling on top (outer side), while giving the advantages of spray bonding on the back (inner) side. The new core material outer surface is found to be 15 times softer than the original core material when measured with a pillow durometer tester with the same flow.

In some embodiments, a zeolite material may be added to the core material during the air-lay process. A known issue with urinary products and collection devices is odor from urea and ammonia. An absorbent core according to the invention herein may include a powered zeolite material which is intermixed with the non-woven fiber materials at the time of formation of the non-woven web, i.e. during an airlay or other process for creating the non-woven core material. Zeolite is a type of mineral that is made up of a framework of silicon, aluminum, and oxygen. This framework acts as a cage and traps other molecules inside of the zeolite. There are many types of zeolites, and each type has a different type of cage structure. A natural zeolite (one that is naturally occurring called clinoptilolite) can be used to capture ammonia and therefore can act as an ammonia absorber. A powdered zeolite material contained within the absorbent core can therefore operate to trap ammonia within urine and reduce odors.

Referring now to FIG. 12, in some embodiments, the finished core assembly 12/112 may be placed into a cylindrical mold 140 and heated and cooled (blossoming) to release and reset the polymer core assembly 112 into a more consistent cylindrical shape which will improve comfort and generally improve consistency in assembly of the core 112 with the housing 114. In some embodiments, the mold 140 may be provided with an arcuate curvature and an internal curved shaping rod 142 to not only round the diameter of the core but also provide it with a predefined shape consistent with the arcuate curvature of the shape retention element generally improving the shape retention of the entire device after assembly.

In some embodiments, ribs 144 may be added to the inside of the blossoming mold 140 to create exterior surface ribs on the core which mimic the current exterior look of the product currently in production.

As explained above, the core assembly 112 and tubing assembly 115 are then secured within the outer housing 114 to complete the assembly.

While there is shown and described herein certain specific structure embodying the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed is:

1. A suction tube assembly for use in a device for collecting urine comprising:
an elongated shape retention element having rounded terminal ends; and
a suction tube body having a suction lumen providing a suction path from a first end thereof to a second end thereof, and a second shape retention lumen receiving the elongated shape retention element therein.

2. The suction tube assembly of claim 1 wherein the shape retention element is formed with a predefined curvature.

3. The suction tube assembly of claim 1 wherein the shape retention element comprises a ductile metal and is bendable to a predefined shape.

4. The suction tube assembly of claim 1 wherein the shape retention element has embossed features on at least a portion of an external surface.

5. The suction tube assembly of claim 1 wherein the suction tube body is generally cylindrical.

6. The suction tube assembly of claim 1, wherein the suction tube body has a D-shaped cross-section with a planar surface and a rounded surface, wherein suction lumen is oriented adjacent the planar surface and wherein the shape retention lumen is oriented adjacent the rounded surface.

7. The suction tube assembly of claim 6 wherein the shape retention element is formed with a predefined curvature.

8. The suction tube assembly of claim 6 wherein the shape retention element comprises a ductile metal and is bendable to a predefined shape.

9. The suction tube assembly of claim 6 wherein the shape retention element has embossed features on at least a portion of an external surface.

10. A device for collecting urine comprising:
a permeable core assembly comprising:
a tubular fluid permeable core having a cylindrical external surface;
a fluid permeable wicking cover disposed on the external surface of the tubular fluid permeable core;
an elongated, capsular-shaped, fluid impermeable housing having a reservoir at a lower end, an outlet port at an upper end, and an elongated window extending at least partially between the lower end and the upper end, the core assembly received within the housing such that the core assembly is disposed within and extends across the elongated window; and
a suction tube assembly extending through an interior of the core assembly and having a first end disposed in the reservoir and a second end exiting the housing through the outlet port, the suction tube assembly comprising,
an elongated shape retention element having rounded terminal ends, and
a suction tube body having a suction lumen providing a suction path from a first end thereof to a second end thereof, and a second shape retention lumen receiving the elongated shape retention element therein.

11. The device of claim 10 wherein the shape retention element is formed with a predefined curvature.

12. The device of claim 10 wherein the shape retention element comprises a ductile metal and is bendable to a predefined shape.

13. The device of claim 10 wherein the shape retention element has embossed features on at least a portion of an external surface.

14. The device of claim 10 wherein the suction tube body is generally cylindrical.

15. The device of claim 10, wherein the suction tube body has a D-shaped cross-section with a planar surface and a rounded surface, wherein suction lumen is oriented adjacent the planar surface and wherein the shape retention lumen is oriented adjacent the rounded surface.

16. The device of claim 15 wherein the shape retention element is formed with a predefined curvature.

17. The device of claim 15 wherein the shape retention element comprises a ductile metal and is bendable to a predefined shape.

18. The device of claim 15 wherein the shape retention element has embossed features on at least a portion of an external surface.
